(19) United States
(12) Patent Application Publication
(10) Pub. No.: US 2025/0268606 A1
(43) Pub. Date: Aug. 28, 2025

(54) Medical Clamping Device

(71) Applicants: Sang In Han, Fairview Heights, IL (US); Eugene Han, Redding, CT (US); Rebecca Han, Oak Park, IL (US)

(72) Inventors: Sang In Han, Fairview Heights, IL (US); Eugene Han, Redding, CT (US); Rebecca Han, Oak Park, IL (US)

(21) Appl. No.: 18/599,694

(22) Filed: Mar. 8, 2024

Related U.S. Application Data
(60) Provisional application No. 63/558,330, filed on Feb. 27, 2024.

(51) Int. Cl. A61B 17/122; A61B 17/12

(52) U.S. Cl. CPC A61B 17/122; A61B 2017/12004

(57) ABSTRACT

A clamping device includes a flexible first part that has a first side wall and a second side wall that is spaced opposite from and is joined to the first side wall. The first side wall and the second side wall are flexible and being configured to move toward one another. The first part includes a locking bar coupled to the first side wall and the second side wall includes a locking protrusion that is configured to engage and lock with the locking bar to close off and lock the first part. The first part has an open unlocked position, in which the locking bar is disengaged with the locking protrusion, for receiving a traumatized digit. The device includes a cushioned second part that lines an inner face of the first part. The cushioned second part includes rounded inner portions configured to support a digit when the device is in a closed locked position for capturing the traumatized digit between the cushioned second part.

### CROSS REFERENCE TO RELATED APPLICATION

The present application claims priority to and the benefit of U.S. patent application Ser. No. 63/558,330, filed Feb. 27, 2024, which is hereby expressly incorporated by reference in its entirety.

### TECHNICAL FIELD

The present disclosure relates to medical devices and more specifically, to a medical clamping device that stops bleeding of a patient's digit (e.g., a finger) in situations where the digit has undergone trauma and is bleeding.

### BACKGROUND

The fingers and toes are richly vascularized and bleed heavily during trauma. Trauma can be in the form of a laceration to the finger or toe or a finger avulsion or similar type of trauma. Tourniquets are often applied to the digits to control and minimize blood loss providing a clear surgical field that allows for delineation of structures, thereby reducing risk for damage to underlying vital structures and increasing the prospects for positive outcomes. As is known in the field, a tourniquet is a constricting device used to control venous and arterial circulation to an extremity for a period of time.

A field obscured by actively oozing blood creates a nearly impossible situation for fully evaluating the presence of joint capsule or tendon injury and foreign body. The “bloodless field” improves precision in repair and reduces risk of inadvertently damaging underlying vital structures such as tendons, etc. There are many types of tourniquets that are intended to provide a bloodless field. One type of product is a ring-like band that constricts blood flow across the entire diameter of the digit.

The present disclosure describes and illustrates an improved clamping device to treat a digit that has undergone trauma and is bleeding.

### SUMMARY

A clamping device according to one embodiment disclosed herein includes a flexible first part that has a first side wall and a second side wall that is spaced opposite from and is joined to the first side wall. The first side wall and the second side wall are flexible and being configured to move toward one another. The first part includes a locking bar coupled to the first side wall and the second side wall includes a locking protrusion that is configured to engage and lock with the locking bar to close off and lock the first part. The first part has an open unlocked position, in which the locking bar is disengaged with the locking protrusion, for receiving a traumatized digit. The clamping device includes a cushioned second part that lines an inner face of the first part. The cushioned second part includes rounded inner portions configured to support a digit when the device is in a closed locked position for capturing the traumatized digit between the cushioned second part.

In one embodiment, a clamping device includes a first part that has a first side wall and a second side wall that is spaced opposite from and is joined to the first side wall. The first side wall and the second side wall are flexible and are configured to move toward one another, wherein the first part includes a locking bar and the second side wall includes a locking protrusion that is configured to engage and lock with the locking bar. The clamping device further includes a cushioned second part that lines an inner face of the first part. The clamping device has an open unlocked position for receiving a traumatized digit and a closed locked position for capturing the traumatized digit between the cushioned second part.

### BRIEF DESCRIPTION OF THE DRAWING FIGURES

FIG. 1 is a front perspective view of the clamping device in a first position;

FIG. 2 is a front perspective view of the clamping device in a second position;

FIG. 3 is a front perspective view of the clamping device in a third position;

FIG. 4 is a front perspective view of the clamping device in a fourth position;

FIG. 5 is a front perspective view of the clamping device in a fifth position;

FIG. 6 is a perspective showing an injured finger;

FIG. 7A a perspective view of the clamping device with the injured finger being clamped;

FIG. 7B is a side view thereof;

FIG. 8 is a perspective view of the clamping device with the injured finger being clamped and a delivery needle;

FIG. 9A a perspective view of the clamping device with the injured finger being clamped; and

FIG. 9B is a side view thereof.

### DETAILED DESCRIPTION OF CERTAIN EMBODIMENTS

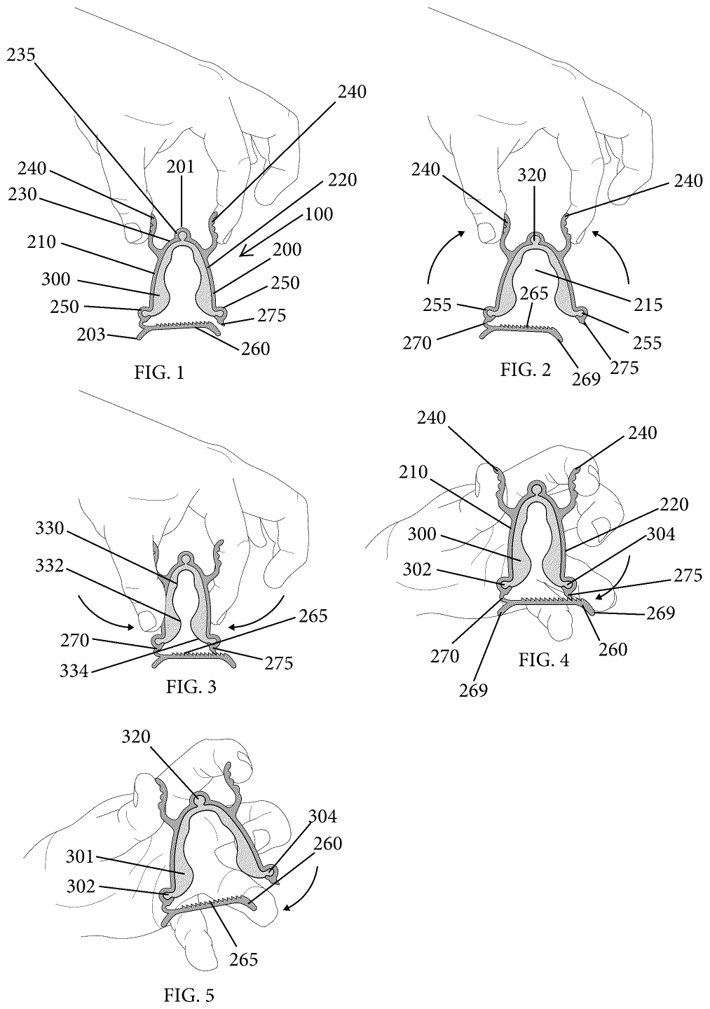

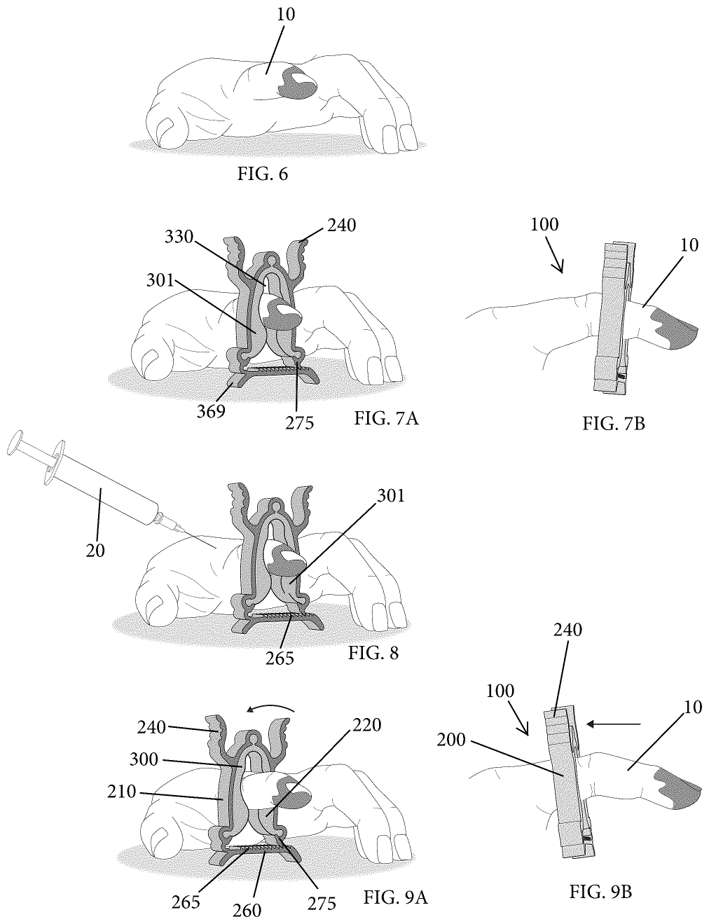

FIGS. 1-9B illustrate a clamping device 100 for use with a digit, such as a finger 10. The clamping device 100 is configured to stop the bleeding of a patient's digit (finger) in situations where the digit has undergone trauma and is bleeding. FIGS. 1-5 show the structure and movements of the clamping device 100, while FIGS. 6-9B show a method of treatment using the clamping device 100.

The clamping device 100 is generally formed of a first part 200 and a second part 300. The first part 200 can be generally thought of as being an outer rigid element and the second part 300 can be generally thought of as being an inner cushioning element. The first part 200 is configured to maintain shape and tension and the second part 300 is a cushioning element that makes contact with the relevant digit.

### First Part 200

As described herein, the first part 200 moves between an open (unlocked) position and a closed (locked) position. The first part 200 has a closed first end 201 and an open second end 203.

The first part 200 includes a first side wall 210 and a second side wall 220 that is joined, in a continuous manner, to the first side wall 210 along a top wall section 230. Between the first side wall 210 and the second side wall 220 there is an open space 215 for receiving the digit. The combined first side wall 210, second side wall 220 and the top wall section 230 generally have a U shape. While the first part 200 is rigid, it also has a degree of flexing as shown in the figures. In particular, the first and second side walls 210, 220 can be considered to be fingers or prongs that are free to flex when a force is applied thereof, whereby, as shown, the first and second side walls 210, 220 can move toward one another or away from one another. For example, the first part 200 can be a molded plastic piece.

Along an inner face of the top wall section 230, there is a coupling channel 235. The coupling channel 235 can be at the apex of the first part 200.

At or proximate the first end 201, the first part 200 includes a pair of outer levers 240 that protrude and extend radially outward. For example, one outer lever 240 is integral to and extends outward from the first side wall 210 and the other outer lever 240 is integral to and extends outward from the second side wall 220. As shown, the pair of outer levers 240 can have curved shapes, including concave sections, to accommodate the thumb or finger of the user and allow a pressing action. The outer levers 240 are rigid but have some degree of flexing. As shown in FIGS. 1 and 2, the outer levers 240 are configured such that when an inward force is applied thereto, it results in opposite motion in the first and second side walls 210, 220 in that the first and second side walls 210, 220 move away from one another. In other words, the first and second side walls 210, 220 open up and the open space 215 between the first and second side walls 210, 220 increases in area.

The outer levers 240 can have a contoured outer surface, such as ribbing, to increase grip.

A bottom end of each of the first and second side walls 210, 220 can include a flange 250 that protrudes outward. The flange 250 can be a rounded flange that is formed so as to define an inner channel 255 that faces inward and is in communication with the open space 215. As described herein, this inner channel 255 is configured to capture the second part 300. The channel 255 can have rounded inner walls. The two flanges 250 and two inner channels 255 are opposite one another.

The bottom end of the second side wall 220 has a locking protrusion 275 that can act as a locking pawl or tooth. The locking protrusion 275 extends downward and is angled outward. The locking protrusion 275 is preferably an integral part of the first part 200.

The first part 200 also includes an elongated toothed locking bar (toothed flange) 260 that is coupled to the bottom of the first side wall 210. The toothed locking bar 260 has a fulcrum 270 about which the locking bar 260 pivots and more specifically, moves between an open position and a closed position. The fulcrum 270 thus represents the connection location between the locking bar 260 and the first side wall 210.

The length of the locking bar 260 is such that the free end thereof extends laterally beyond the flange 250 associated with the second side wall 220.

The locking bar 260 has teeth 265 formed along an upper surface of the locking bar 260. The teeth 265 are formed at an angle along the upper surface that is complementary to the angle and structure of the locking protrusion 275. In other words, the locking protrusion 275 contacts and engages the teeth 275 and in particular, is positioned between two teeth 275 resulting in the locking bar 260 being coupled to the second side wall 220. The locking bar 260 is thus engaged and locked to the second side wall 220, resulting in the space 215 being closed off. The locking bar 260 can have a pair of downwardly extending legs 269. The legs 269 can be located at or near the ends of the locking bar 260 and extend laterally outward. The legs 269 allow the device 100 to stand up (be upright) on a flat surface.

FIGS. 2 and 3 illustrate the locking action and movements of the device 100. For example, in FIG. 3, the user is pressing inward the first and second side walls 210, 220 which causes the locking protrusion 275 to also move inward and the user can direct the locking protrusion 275 into and between teeth 275 formed on the locking bar 260 resulting again in the locking action.

### Second Part 300

As mentioned, the second part 300 is a cushioning element that makes contact with the relevant digit. The second part 300 has a shape that is complementary to the inside of the first part 200 since the second part 300 lines the inner face of the first part 200. The second part 300 lines the inner surface of the first part 200.

The second part 300 is an elongated structure that has a first end 302 and an opposite second end 304. The first end 302 has a shape that is received into and captured within the inner channel 255 of the flange 250 that is part of the first side wall 210 and similarly, the second end 304 of the second part 300 is received within and captured within the inner channel 255 of the flange 250 that is part of the second side wall 220. This secures the two opposite ends of the second part 300 to the first part 200. To secure the center region of the second part 300 to the first part 200, a center flange or protrusion or bead 320 of the second part 300 is received into the coupling channel 235. This now results in the entire second part 300 lining the inner surface of the first part 200 since the second part 300 is secured to the first part 200 at three distinct locations, namely, the two ends and center. However this friction fit can be reversed and with applied force, the second part 300 can be detached from the first part 200.

As shown, the inner face of the second part 300 can be contoured in view of its intended use. For example, as shown, the hollow interior of the second part 300 can have an enlarged upper space 330, a smaller waist 332 below the enlarged upper space 330 and an outwardly flared lower space 334. In particular, the second part 300 can include a pair of inwardly directed rounded portions 301 that act to support the contained finger 10 when the device 100 is closed. These rounded portions 301 define the waist 332 and when contained the digit (finger 10) is located within the enlarged upper space 330 and rests on the waist 332.

The construction of the clamping device 100 allows the patient to rest their hand while the traumatized digit remains raised for care.

The second part 300 is formed of a cushioned material, such as silicone.

### Method of Treatment (FIGS. 6-9)

FIG. 6 generally shows trauma at the end of one finger 10.

With additional reference to FIGS. 1-5, in its initial state (FIG. 1), the clamping device 100 is in the unlocked position and its aperture (space 115) can be adjusted by compressing the two outer levers 240 towards each other as shown in FIG. 2. As mentioned, to lock the device 100 in place, the user squeezes the first and second side walls 210, 220 and the locking protrusion 275 is brought into engagement with the teeth 265. As this locking action occurs, the second part 300 contacts and applies pressure to the contained digit (finger 10) as shown in FIGS. 3, 7A and 7B.

After the device 100 is secured and the bleeding is controlled, the traumatized area can now be worked on.

When no longer requires, the device 100 can be unlocked by lowering the locking bar 260, thereby disengaging the locking protrusion 275 from the teeth 265 and unlocking the device 100. Once unlocked, the second end of the device 100 is open and the traumatized finger can be removed from the device 100.

As shown in FIGS. 9A and 9B, if required, the device 100 may be adjusted to further facilitate care.

The present device 100 provides a number of advantages over traditional clamping devices. For example, in ring-like devices, oftentimes the clamping device must pass over the damaged area, causing possible extreme discomfort to the patient, and possibly causing additional damage. The device 100 need not make contact with the affected digit in the same manner, as its aperture (space 215) can be adjusted to accommodate for many injuries to the finger. Therefore, the case of application reduces risk of additional trauma in its application and removal. The pressure exerted by the device 100 is inherently adjustable due to the array of locking teeth 265. This is important as the caretaker need only apply the minimum necessary pressure to control bleeding, which as the added benefit of minimizing risk due to blocking blood circulation. In practice, the caretaker may need to check the blood circulation and tissue perfusion while tending to the traumatized digit. Using other tourniquet devices, this would require application of multiple device unites because they are unable to release pressure during the procedure. In contrast, since the device 100 is adjustable, multiple sized devices are not required. Another adjacent is shown in FIGS. 7A and 7B in that, when applied, the device 100 allows for physical support of the patient's fingers and hand to facilitate application of sterile- strips or cauterization. FIG. 8 shows that when the device 100 is applied, local anesthetics can be applied as by using a needle 20. In both locking the device 100, as well as releasing the device, a minimum of one hand may be used if operating conditions allow, whereas other such devices require two hands.

Unlike other tourniquet-like devices, the present clamping device 100 involves controlling bleeding by targeting the main arteries and veins, without blocking peripheral circulation. In traditional products, pressure is applied around the entire digit, whereas, in the present clamping device 100, pressure is applied specifically to arteries and veins due to the unique configuration of the clamping device 100. The present clamping device 100 thus blocks blood flow by compressing arteries, as opposed to entire digit constriction. This results from the construction and shape of the clamping device.

The present clamping device 100 also allows for a variable range of pressures to facilitate care.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

What is claimed is:

1. A clamping device comprising:
a first part that has a first side wall and a second side wall that is spaced opposite from and is joined to the first side wall, the first side wall and the second side wall being flexible and being configured to move toward one another, wherein the first part includes a locking bar and the second side wall includes a locking protrusion that is configured to engage and lock with the locking bar; and
a cushioned second part that lines an inner face of the first part;
wherein the clamping device has an open unlocked position for receiving a traumatized digit and a closed locked position for capturing the traumatized digit between the cushioned second part.

2. The clamping device of claim 1, wherein the first part has a pair of outer levers at a top end of the first part that when compressed toward one another are configured to separate the first and second side walls.

3. The clamping device of claim 1, wherein the locking bar is attached to the first side wall at a fulcrum.

4. The clamping device of claim 3, wherein the locking bar comprises an elongated cantilevered structure that has a free end proximate the second side wall.

5. The clamping device of claim 1, wherein the locking bar has teeth formed along a top surface thereof and the locking protrusion engages the teeth to lock the locking bar to the second side wall.

6. The clamping device of claim 1, wherein the locking bar has a pair of downwardly extending legs on which the device can stand upright.

7. The clamping device of claim 1, wherein the second part is attached to the first part at at least three locations.

8. The clamping device of claim 7, wherein the second part is attached to the first part at a first end, a second end and a center of the second part.

9. The clamping device of claim 1, wherein the second part is disposed above the locking bar.

10. The clamping device of claim 1, wherein the second part is contoured and includes an enlarged upper opening, a narrow waist, and a flared lower opening.

11. The clamping device of claim 1, wherein the first part includes a first flange at a bottom of the first side wall and a second flange at a bottom of the second side wall, the first flange including a first inner channel and the second flange including a second inner channel.

12. The clamping device of claim 11, wherein the locking protrusion is formed adjacent the second inner channel.

13. The clamping device of claim 11, wherein the second part is received at a first end within the first inner channel and is received at a second end within the second inner channel.

14. A clamping device comprising:
a flexible first part that has a first side wall and a second side wall that is spaced opposite from and is joined to the first side wall, the first side wall and the second side wall being flexible and being configured to move toward one another, wherein the first part includes a locking bar coupled to the first side wall and the second side wall includes a locking protrusion that is configured to engage and lock with the locking bar to close off and lock the first part, the first part having an open unlocked position, in which the locking bar is disengaged with the locking protrusion, for receiving a traumatized digit; and
a cushioned second part that lines an inner face of the first part, the cushioned second part including rounded inner portions configured to support a digit when the device is in a closed locked position for capturing the traumatized digit between the cushioned second part.
